SEQUENCE LISTING (2 sequences)

SEQ ID NO: 1 - protein, 485 aa, artificial sequence
  synthetic construct
ASTNGTMMQYFEWYLPNDGNHWTRLQNDAANLSSKGVTAVWIPPAYKGTSQNDVGYGAYDLYDLGEFNQKGTVRTKYGTKAQLKSAIAELHKQNIHVYGDVVMNHKGGADFTETVTAVEVDPNNRNRETSGDYQISAWTGFTFPGRGNTYSPFKWNWYHFDGTDWDESRKINRIYKFRGTGKAWDWEVSSENGNYDYLMYADIDYEHPDVINEMKKWGTWYAKELNLDGFRLDAVKHIKHDYLRDWVTHVRNQTGKELFTVAEYWQNDIGQLNNYLAKTNYNQSVFDAPLHYHFHYASNSNGNYDMRNILKGTVMESNPRLAVTLVENHDSQPGQSLESVVSTWFKPLAYAFILTRAEGYPSVFYGDYYGTKGNSNYEIPSLKTQLDPILTARKNYAYGLQRDYIDHWDIIGWTREGEAARDKSGLATLITDGPGGYKWMEVGKQNAGEVWYDITGNRKETVTINNDGWGQFYVNGGSVSIYVQK

SEQ ID NO: 2 - protein, 353 aa, artificial sequence
  synthetic construct
AEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2025/0285710 A1
(43) Pub. Date: Sep. 11, 2025

(54) SYSTEMS AND METHODS FOR GENERATING DIVERGENT PROTEIN SEQUENCES

(71) Applicants: Michael LISZKA, San Diego, CA (US); BASF SE, Ludwigshafen am Rhein (DE)

(72) Inventors: Michael LISZKA, San Diego, CA (US); Zachary David MILES, San Diego, CA (US)

(21) Appl. No.: 18/554,895

(22) PCT Filed: Apr. 4, 2022
(86) PCT No.: PCT/US22/23288

§ 371 Date: Oct. 11, 2023

Related U.S. Application Data
(60) Provisional application No. 63/176,624, filed on Apr. 19, 2021.

(51) Int. Cl. G16B 35/00; G16B 15/20; G16B 15/30

(52) U.S. Cl. CPC G16B 35/00; G16B 15/20; G16B 15/30

(57) ABSTRACT

Disclosed herein are computer-implemented systems and methods for generating functional protein sequences using a library of protein fragments.

### CROSS-REFERENCE TO RELATED APPLICATIONS

This application is a U.S. National Phase Application of International Patent Application No. PCT/US22/23288, filed Apr. 4, 2022, which claims priority to U.S. Patent Application No. 63/176,624, filed Apr. 19, 2021, each of which is hereby incorporated by reference herein.

### REFERENCE TO AN ELECTRONIC SEQUENCE LISTING

The contents of the electronic sequence listing (txt_27843_2457_sequence_listing.txt; Size: 7,606 bytes; and Date of Creation: Mar. 13, 2024) are herein incorporated by reference in their entirety.

### FIELD OF TECHNOLOGY

The present disclosure relates to the field of biotechnology, and, more specifically, to computer-implemented systems and methods for generating functional protein sequences using a library of protein fragments.

### BACKGROUND

The three-dimensional (3D) structure and function of a protein is dictated by its amino acid sequence. Proteins similar in amino acid sequence tend to fold into similar structures and often have a similar function. The primary structure of a protein refers to the sequence of the amino acids in the polypeptide chain. Peptide bonds can only form linear structures and proteins do not contain branching chains. The secondary structure of a protein refers to the localized spatial and repetitive arrangements of its polypeptide chain (e.g., alpha-helices, beta-sheets), which are generally held together by hydrogen bonds. Tertiary structure describes the complete 3D architecture of the protein. The driving forces that allows proteins to fold are the hydrogen bond interactions within the backbone and between the side chains, Van der Waals forces, and principally the interaction of hydrophobic side chains within the core of the folded protein.

Computational methods have been developed to predict the 3D structure of proteins. For example, homology modeling techniques may be used to generate a predicted protein structure using a previously-crystalized structure that shares a high degree of full-length sequence identify (e.g., 90%) as a template. Such methods operate based on the theory that highly similar polypeptide sequences are likely to share a highly similar 3D structure. Research in this area has also explored the possibility of using fragment libraries to assemble a protein. For example, the Rosetta software package includes a comparative modeling application that can predict the tertiary structure of a protein of interest using a library of fragments generated from proteins for which a crystal structure has been published in the Protein Databank (or other repositories). See, e.g., Srivatsan et al. “Structure prediction for CASP8 with all-atom refinement using Rosetta.” Proteins 77 Suppl 9:89-99 (2009) and Chivian et al. “Homology modeling using parametric alignment ensemble generation with consensus and energy-based model selection.” Nucleic Acids Research 34(17)e112 (2006), the contents of which are incorporated herein by reference in their entirety. As such, Rosetta's comparative modeling approach relies upon the same paradigm applied to homology modeling generally, i.e., selection of template which shares a high degree of sequence identity with portions of the input protein sequence being modeled, on the basis that high sequence identify will result in a highly similar structure.

As exemplified by Rosetta and similar tools, known methods for protein modeling have focused on the paradigm that conserved sequences produce conserved structures. Such tools are useful for predicting the tertiary structure of arbitrary polypeptide sequences. However, they are limited in the sense that they do not generate new polypeptide sequences, merely structural information for known sequences. Researchers interested in studying the relationship between protein structure and function have therefore been generally limited to proteins for which crystal structures are available, and predicted structures generated using homology modeling and other similar techniques. This limited dataset samples very little of the vast conformational space available to proteins. For example, a 200 amino acid protein has 20200 possible polypeptide sequences (assuming that the protein only includes the 20 standard amino acids). Each of these sequences may adopt a different fold based on its constituent amino acids, and many of these sequences will encode proteins that have no useful functionality with respect to in vivo or industrial processes. Given this large search space, it would be impractical for researchers to generate and study random protein sequences. Indeed, in order to study this conformational space, new tools are needed to generate novel polypeptide sequences which are likely to encode functional proteins.

### SUMMARY OF VARIOUS ASPECTS OF THE INVENTION

To address these and other needs, aspects of the present disclosure describe methods and systems for generating divergent protein sequences which are likely to encode functional proteins (e.g., enzymes). Such methods may, e.g., use the three-dimensional structure of a protein of interest and of fragments of previously-crystalized protein structures, to generate a novel protein sequence that diverges from the polypeptide sequence of the protein of interest while retaining the same or similar functionality (e.g., enzymatic activity) compared to the protein of interest.

In one exemplary aspect, such methods may comprise a) receiving structural data for a protein of interest; b) generating a first library of fragments using the structural data, wherein the first library of fragments comprises fragments of the protein of interest; c) selecting one or more template proteins; d) generating a second library of fragments, wherein the second library of fragments comprises fragments of each of the one or more template proteins; e) comparing at least one fragment in the first library of fragments against one or more fragments in the second library of fragments; f) selecting a replacement fragment for at least one of the fragments in the first library of fragments, based on the comparison; and g) generating a divergent protein sequence, wherein the divergent protein sequence comprises at least one replacement fragment.

In some aspects, the structural data comprises a three-dimensional structure of the protein of interest. For example, the structural data may comprise a protein data bank (PDB) file containing coordinates representing a three-dimensional structure of the protein of interest.

In some aspects, the first library of fragments is generated by parsing the structural data into a series of segments and extracting coordinates for each segment from the structural data. For example, the segments may comprise at least, at most, or exactly 2, 3, 4, 5, 6, 7, 8, 9, 10, 11, 12, 13, 14, 15, 16, 17, 18, 19, or 20 amino acids in length. In some aspects, the structural data may be parsed into a series of segments having a length that falls within a range with endpoints defined by any of the foregoing values (e.g., a length of between 5-15 or 10-20 amino acids).

In some aspects, the structural data may be parsed into a series of segments of uniform length. In other aspects, the structural data may be parsed into a series of segments of uniform length spanning a majority of the protein of interest, plus an additional segment having a different length (e.g., to account for the total length of the protein of interest not being a multiple of a preferred segment size).

In some aspects, the first library of fragments comprises coordinates representing a three-dimensional structure for each of a plurality of fragments of the protein of interest. Fragments in the first and/or second library of fragments may comprise, e.g., at least, at most, or exactly 2, 3, 4, 5, 6, 7, 8, 9, 10, 11, 12, 13, 14, 15, 16, 17, 18, 19, or 20 amino acids in length. For example, the first and/or second library of fragments may comprise fragments of at least, at most, or exactly 6 or 8 amino acids in length. In some aspects, the first and/or second library of fragments comprises fragments having a length that falls within a range with endpoints defined by any of the foregoing values (e.g., a length of between 5-15 or 10-20 amino acids).

In some aspects, the one or more template proteins comprise proteins for which a crystal structure is available. In some aspects, the one or more template proteins are selected based upon one or more parameters, comprising: a) a sequence identity threshold parameter; b) an enzyme classification parameter; c) the presence of one or more protein domains; and/or d) a superimposition parameter reflecting a degree of local or global fit when a 3D structure of the template protein, or a portion thereof, is superimposed on a 3D structure of the protein of interest, or a portion thereof. In some aspects, the one or more template proteins are selected based upon a maximum sequence identity threshold, where in the maximum sequence identity comprise at most 10, 20, 30, 40 50, 60, 70, 80, or 90% full length sequence identity compared to the protein of interest.

In some aspects, structural data is provided for each of the template proteins and the second library of fragments is generated by parsing the structural data for each of the template proteins into a series of segments and extracting coordinates for each segment from the structural data. In some aspects, the segments may, e.g., comprise at least, at most, or exactly 2, 3, 4, 5, 6, 7, 8, 9, 10, 11, 12, 13, 14, 15, 16, 17, 18, 19, or 20 amino acids in length. In some aspects, the structural data may be parsed into a series of segments having a length that falls within a range with endpoints defined by any of the foregoing values (e.g., a length of between 5-15 or 10-20 amino acids).

In some aspects, the comparing step comprises generating a pairwise alignment score for at least one fragment in the first library of fragments against one or more of the fragments in the second library of fragments. In some aspects, the comparing step comprises generating a pairwise alignment score for each fragment in the first library of fragments against each fragment in the second library. For example, the comparison step may be performed as an iterative process whereby each fragment in the first library is compared against one or more fragments in the second library, starting from the fragment representing the N-terminus of the protein of interest and ending with the fragment representing the C-terminus of the protein of interest. The pairwise alignment score may be based on sequence identity percentage, sequence similarity percentage, and/or a three-dimensional alignment of the fragment in the first library of fragments against the respective fragment in the second library of fragments. For example, the pairwise alignment score may be based on a three-dimensional alignment of the backbone atoms of the aligned fragments (e.g., using the mean Euclidean distance of one or more corresponding backbone and/or sidechain atoms). In some aspects, replacement fragments are selected for fragments in the first library of fragments based upon pairwise alignment scores, wherein each pairwise alignment score compares the three-dimensional alignment of the fragment in the first library against a fragment in the second library of fragments. The replacement fragment may be a fragment selected from the second library of fragments which displays the highest pairwise alignment score compared against the respective fragment in the first library of fragments. In some aspects, the methods described herein may also comprise a step of generating a predicted protein structure and/or a model quality score for the divergent protein sequence.

In another exemplary aspect, the disclosure provides a system for generating divergent protein sequences, comprising a processor configured to: a) receive structural data for a protein of interest; b) generate a first library of fragments using the structural data, wherein the first library of fragments comprises fragments of the protein of interest; c) select one or more template proteins; d) generate a second library of fragments, wherein the second library of fragments comprises fragments of each of the one or more template proteins; e) compare at least one fragment in the first library of fragments against one or more fragments in the second library of fragments; f) select a replacement fragment for at least one of the fragments in the first library of fragments, based on the comparison; and g) generate a divergent protein sequence, wherein the divergent protein sequence comprises at least one replacement fragment. In other aspects, such systems may comprise a process that is further configured to perform any of the methods (or steps thereof) described herein.

In another exemplary aspect, the disclosure provides a non-transitory computer-readable medium storing thereon computer-executable instructions for generating divergent protein sequences. Such computer-executable instructions may comprise instructions for performing any of the methods (or steps thereof) described herein.

In another exemplary aspect, the disclosure provides divergent protein sequence produced by a computer, comprising a processor configured to: a) receive structural data for a protein of interest; b) generate a first library of fragments using the structural data, wherein the first library of fragments comprises fragments of the protein of interest; c) select one or more template proteins; d) generate a second library of fragments, wherein the second library of fragments comprises fragments of each of the one or more template proteins; e) compare at least one fragment in the first library of fragments against one or more fragments in the second library of fragments; f) select a replacement fragment for at least one of the fragments in the first library of fragments, based on the comparison; and g) generate the divergent protein sequence, wherein the divergent protein sequence comprises at least one replacement fragment; wherein the divergent protein sequence shares at most 10% full-length sequence identity with the sequence of the protein of interest. In some aspects, the divergent protein sequence shares at most 20, 30, 40, 50, 60, 70, 80, or 90% full-length sequence identity with the sequence of the protein of interest.

In some aspects, the divergent protein sequence shares at most 10, 20, 30, 40, 50, 60, 70, 80, or 90% full-length sequence identity with the sequence of the protein of interest and the protein of interest comprises an enzyme; and the divergent protein encodes a protein that maintains at least substantially equivalent enzymatic activity compared to the protein of interest. For example, the divergent protein sequence may maintain ±10% enzymatic activity compared to the protein of interest, when measured using the same assay and identical test conditions.

The above simplified summary of exemplary aspects serves to provide a basic understanding of the present disclosure. This summary is not an extensive overview of all contemplated aspects, and is not intended to identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more exemplary aspects of the present disclosure and, together with the detailed description, serve to explain their principles and implementations.

FIG. 1 is a flow diagram showing an exemplary method for generating a divergent protein sequence, in accordance with aspects of the present disclosure.

FIG. 2 is a flow diagram showing another exemplary method for generating a divergent protein sequence, in accordance with aspects of the present disclosure.

FIG. 3 is chart showing the properties of exemplary replacement fragments selected for inclusion in a divergent protein sequence produced in accordance with aspects of the present disclosure.

FIG. 4 illustrates an example of a general-purpose computer system on which aspects of the present disclosure can be implemented.

### DETAILED DESCRIPTION

Exemplary aspects are described herein in the context of a method, system and computer program product for generating divergent protein sequences using protein fragment libraries. Other exemplary aspects of the disclosure include divergent protein sequences produced, e.g., using such methods and systems. In some aspects, the divergent protein sequences described herein may encode a protein that displays substantially equivalent or improved functionality, as compared to the protein of interest used as a baseline to generate the given divergent protein sequence. For example, the protein of interest may be an enzyme, and the divergent protein sequence may retain the same enzymatic activity, e.g., at a level that is ±10%, ±20%, ±30%, ±40%, ±50%, ±60%, ±70%, ±80%, or ±90% compared to the activity level of the protein of interest, when measured using the same assay and identical test conditions. In some aspects, the divergent protein sequence may encode a protein that has an improved activity level (e.g., at least 10%, 20%, 30%, 40%, 50%, 60%, 70%, 80%, or 90% higher) as measured using the same assay and identical test conditions. The methods, systems, and products described herein provide divergent protein sequences that may be used as alternatives for known proteins which may be used to further study the conformational space available to proteins. Moreover, these divergent protein sequences may be useful in cases where, e.g., a desired protein is unavailable due to manufacturing or supply constraints, or in cases where the sequence of a given protein of interest is proprietary in nature.

Those of ordinary skill in the art will realize that the following description is illustrative only and is not intended to be in any way limiting. Other aspects will readily suggest themselves to those skilled in the art having the benefit of this disclosure. Reference will now be made in detail to implementations of the exemplary aspects as illustrated in the accompanying drawings. The same reference indicators will be used to the extent possible throughout the drawings and the following description to refer to the same or like items.

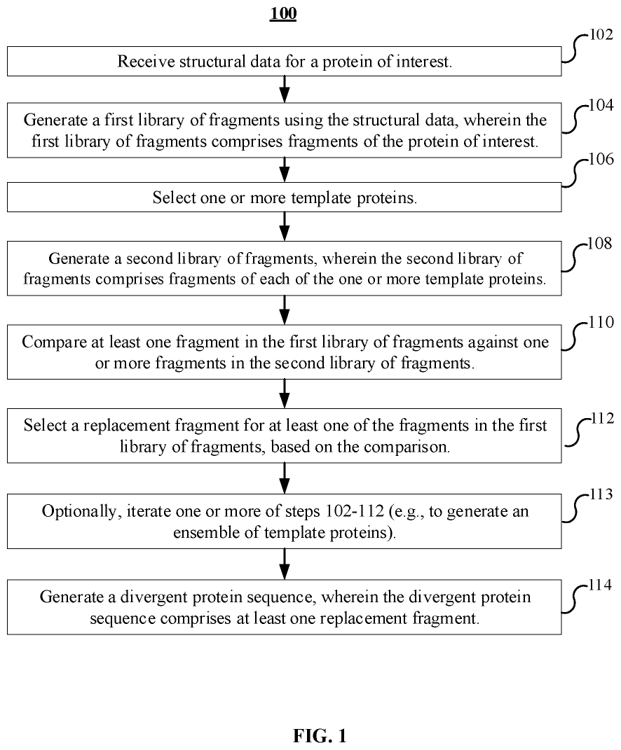

FIG. 1 is a flow diagram of an exemplary method 100 for generating a divergent protein sequence, in accordance with aspects of the present disclosure. At 102, method 100 comprises the step of receiving structural data for a protein of interest. In some aspects, the structural data comprises a three-dimensional structure of the protein of interest. For example, the structural data comprises a protein data bank (PDB) file containing coordinates representing a three-dimensional structure of the protein of interest. The structural data may, e.g., comprise coordinates representing the backbone and/or sidechain atoms of amino acids which form the polypeptide sequence of the protein of interest. In some aspects, the structural data may comprise coordinates representing the backbone and/or sidechain atoms of all amino acids present in the polypeptide sequence of the protein of interest (e.g., a complete structure). In other aspects, the structural data may comprise coordinates representing the backbone and/or sidechain atoms of only some of the amino acids (e.g., a partial structure). It is understood that partial crystal structures are available for some proteins (e.g., publicly accessible protein structure databases include structures for full-length proteins, as well as structures for individual domains or segments of various proteins). In some aspects, the structural data may also include polypeptide sequence data for the protein of interest, or a portion thereof. For example, protein structures encoded in the PDB file format typically include a field that lists the amino acid sequence of the protein structure represented in the file.

At step 104, a first library of fragments is generated using the structural data, wherein the first library of fragments comprises fragments of the protein of interest. For example, the first library of fragments may be generated by parsing the structural data into a series of segments, and extracting coordinates for at least some of the segments from the structural data. In some aspects, coordinates are extracted for each segment. The segments may be of uniform length. In other aspects, the structural data may be parsed into a series of segments of uniform length spanning a majority of the protein of interest, plus an additional segment having a different length (e.g., to account for the total length of the protein of interest not being a multiple of a preferred segment size). Any arbitrary segment size may be used. For example, the segments may comprise at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 amino acids in length (e.g., at least, at most, or exactly 6 or 8 amino acids in length). The segments may alternatively be of a size within a range with endpoints defined by any combination of the foregoing values (e.g., a size of 15-25 amino acids).

A protein structure encoded in a PDB file may be used to generate a first library of fragments of the protein of interest using the structural information stored in this file type. In some cases, a user may select only a portion of the polypeptide sequence of the protein of interest as a basis for the first library of fragments. For example, the user may be prompted, e.g., by software implementing the methods described herein. Such embodiments are advantageous in that a user may only desire to modify a portion of a given protein of interest (e.g., to generate a sequentially divergent active site, binding site, or motif of interest) while retaining the sequence of other portions of the protein of interest.

At step 106, one or more template proteins are selected. A template protein may comprise a protein for which a partial or complete structure is available. In some aspects, the template protein comprises a protein for which a full-length crystal structure is available (e.g., from a public database or private repository). It is understood that a high-resolution crystal structure may be preferred for some applications. However, a low-resolution crystal structure, or even a modeled structure (e.g., a predicted structure generated using homology, comparative, ab initio, or de novo modeling) may be sufficient for many cases. As such, it is envisioned that in some aspects, the methods described herein may further comprise a step of generating a modeled protein structure for use as a template structure. Furthermore, the present methods may include a step of generating a partially modeled structure (e.g., by predicting the structure of one or more portions of a protein for which only a partial crystal structure is available).

The one or more template protein may be selected using various parameters. For example, the one or more template proteins may be selected based upon a minimum or maximum sequence identity threshold, measured locally or for the full-length. For example, a template protein may be selected based on a sequence identity maximum sequence identity of at most 10, 20, 30, 40 50, 60, 70, 80, or 90% full length sequence identity compared to the protein of interest. The use of a maximum sequence identity threshold as a selection parameter ensures that fragments generated from the template protein display a sufficient degree of divergence from the sequence of the protein of interest.

In some aspects, a template protein may be selected based on: an enzyme classification parameter and/or the presence of one or more protein domains or specific amino acids. For example, a search for suitable template proteins may be limited to a search for proteins for which a crystals structure is available, which are classified as enzymes, or classified within a specific family or group of enzymes (e.g., proteases). In some aspects, the selection criteria may require the presence of particular domains, folds, or other structural motifs (e.g., a serine protease domain).

In some aspects, the selection of a template protein may be based on a superimposition parameter reflecting a degree of local or global fit when a 3D structure of the template protein, or a portion thereof, is superimposed on a 3D structure of the protein of interest, or a portion thereof. For example, candidate template proteins may be subjected to a 3D alignment that evaluates the average distance (e.g., root mean squared stance, RMSD) of one or more backbone atoms when the structure of the protein of interest and the template structure are aligned. Various algorithms and programs for aligning the 3D structure of two or more proteins are known in the art, such as the TM-Align program. See, e.g., Zhang et al. “TM-align: A protein structure alignment algorithm based on TM-score,” Nucleic Acids Research, 33:2302-2309 (2005), the entire contents of which is hereby incorporated by reference.

In some aspects, a template protein may be selected on the basis of any combination of the parameters described herein. For example, an initial batch of candidate template proteins may be selected based on a maximum sequence identity threshold, and this set of candidates may be filtered based upon a superimposition parameter, an enzyme classification parameter and/or the presence of one or more protein domains or specific amino acids, to arrive at one or more template proteins finally selected for use in the present methods. It is envisioned that a template protein may be selected based on a single scoring function (e.g., which accounts for one or more of the parameters described herein) or based on an iterative process whereby individual parameters are assessed sequentially, gradually reducing the set of candidate structures.

At step 108, a second library of fragments is generated, wherein the second library of fragments comprises fragments of each of the one or more template proteins. This process is similar to that of step 104, which generated the first library of fragments. In some aspects, structural data is provided for each of the template proteins and the second library of fragments is generated by parsing the structural data for each of the template proteins into a series of segments and extracting coordinates for each segment from the structural data. In some aspects, the segments may, e.g., comprise at least, at most, or exactly 2, 3, 4, 5, 6, 7, 8, 9, 10, 11, 12, 13, 14, 15, 16, 17, 18, 19, or 20 amino acids in length. In some aspects, the structural data may be parsed into a series of segments having a length that falls within a range with endpoints defined by any of the foregoing values (e.g., a length of between 5-15 or 10-20 amino acids).

At step 110, at least one fragment in the first library of fragments is compared against one or more fragments in the second library of fragments. This comparison may be performed, e.g., by generating a pairwise alignment score for at least one fragment in the first library of fragments against one or more of the fragments in the second library of fragments. This pairwise alignment score may be generated based upon sequential or structural information. For example, a 3D structural alignment of the at least one fragment in the first library of fragments and one or more fragments in the second library of fragments may be generated, and a superimposition score may be determined (e.g., an average RMSD of one or more backbone atoms of the aligned residues). With respect to sequence information, a pairwise alignment may be used to evaluate and score the fragment pairs, e.g., by taking into account whether an aligned residue is identical or similar, and/or based upon differences in the physiochemical properties of the fragments (e.g., total charge, net charge, or the number of hydrophobic, aromatic, or neutral polar residues). In some aspects, the comparison step may take into account any combination of these parameters, e.g., as a single aggregate score or by determining scores for one or more discrete parameters (which may optionally be weighted differently) and calculating a summed score. In some aspects, this comparison step may be performed iteratively, such that each fragment in the first library of fragments is compared to a plurality of fragments selected from or spanning across each template protein.

At step 112, a replacement fragment is selected for at least one of the fragments in the first library of fragments, based on the comparison (e.g., based on a score determined during the preceding comparison step. In some aspects, fragments will be selected for all of the fragments in the first library of fragments, whereas in others only one fragment, or a plurality of fragments, are selected. In some aspects, fragments are selected based upon the mean Euclidean distance between one or more atoms in the replacement fragment as compared to the fragment being replaced. In some aspects, the selection may take into account the edit distance (the minimum number of operations required to transform the amino acid sequence of the original fragment into the amino acid sequence of the replacement fragment). In some aspects, the selection may be based on the mean Euclidean distance (e.g., of backbone atoms in the fragments after a rigid optimal structural alignment) and/or on a minimum edit distance (e.g., 1, 2, 3, 4, 5, 6, 7, 8, 9, or 10 operations).

Optionally, at step 113 one or more of steps 102-112 may be iterated any number of times, e.g., to generate an ensemble of templates of the protein of interest. In some aspects, different parameters may be used in each iteration, or in a subset of the iterations (e.g., different segment size parameters, or a different sequence identity threshold, may be used). For example, steps 102-110 may be iterated and replacement fragments may be selected at step 112 from the ensemble of templates generated by the iteration of steps 102-110. In some aspects, steps 102-112 may be iterated, e.g., with a replacement fragment from each template selected at step 112 before the next round of iteration. It is understood that the number of iterations and the steps selected for iteration may be varied as desired for a given implementation.

Finally, at step 114, a divergent protein sequence is generated, wherein the divergent protein sequence comprises at least one replacement fragment. The divergent protein sequence may be generated, e.g., by extracting sequence information from each replacement fragment and then inserting the extracted sequence information into the corresponding location in the polypeptide sequence of the protein of interest. For example, a hypothetical protein of interest may comprise a 280 amino acid polypeptide sequence, and be used as a baseline sequence in a method according to the disclosure which is configured to use a fragment size of 20 amino acids. Such a method could result in the generation of up to 14 replacement fragments (e.g., 14 fragments, each 20 amino acids in length), assuming that the entire protein of interest has been selected for analysis. In this case, the method may result in the selection of replacement fragments for less than all of the positions, e.g., based on scoring thresholds or other parameters evaluated during the comparison step. It is possible that replacement fragments may only be selected for the positions spanning 41-60 and 101-120 of the polypeptide sequence of the protein of interest. The divergent protein sequence may thus be generated by replacing the amino acid sequences originally found in these two segments, with amino acid sequence extracted from these two respective replacement fragments, to produce a new 280 amino acid polypeptide sequence. In some aspects, a divergent protein sequence may comprise only a portion of the polypeptide of interest. In this case, a sequence comprising the segment spanning position 1-60 would also be a divergent protein sequence (i.e., including the sequence found at position 1-40 of the original polypeptide sequence plus the sequence of the replacement fragment inserted from position 41-60).

In some aspects, a method according to the disclosure may further comprise validating the generated divergent protein sequence. For example, the generated sequence may be expressed in a suitable host (e.g., the source of the original protein of interest, or any other suitable expression system) and evaluated to determine whether it possesses any functionality (e.g., the functionality of the protein of interest). In some aspects, validation may comprise determining whether the generated protein of interest maintains substantially equivalent or improved enzymatic activity compared to the protein of interest. As used herein, “substantially equivalent” enzymatic activity is defined as ±10% activity compared to the protein of interest as measured using the same assay and identical test conditions.

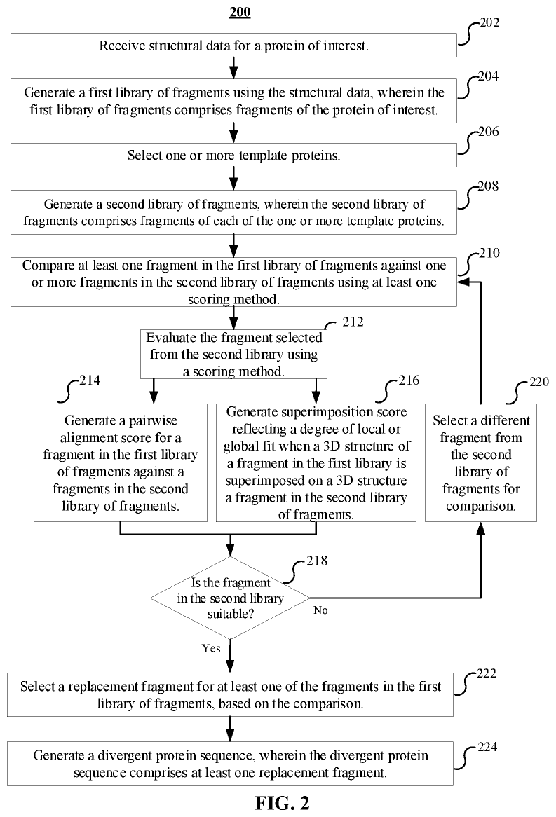

FIG. 2 is a flow diagram of another exemplary method 200 for generating a divergent protein sequence, in accordance with aspects of the present disclosure. This example illustrates exemplary parameters that can be used to score a given fragment selected from the second library (e.g., at steps 212-218). Sequence and/or structural information or properties can be used to select a potential replacement fragment. These parameters are described in further detail above in the description of FIG. 1. As illustrated by this figure, if a given fragment is found not to be suitable at step 218 (e.g., due to having a low score when evaluated using one or more scoring methods, the method may return to the comparison step 210, allowing for multiple fragments to be evaluated as an iterative process, until suitable fragments are identified or until the entire second library of fragments is evaluated.

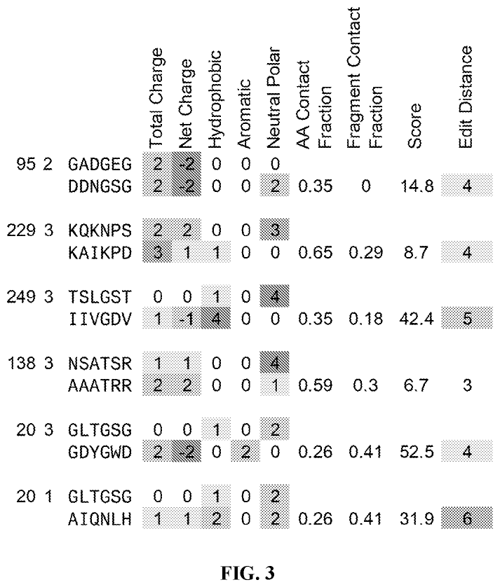

FIG. 3 is chart showing the properties of exemplary replacement fragments selected for inclusion in a divergent protein sequence. In this case, an exemplary method according to the disclosure was validated using the protease subtilisin as a protein of interest. A crystal structure for B. subtilis subtilisin (1ST3) was obtained from a publicly accessible protein database. Structural information and sequential information were extracted from the PDB and used to search for template proteins. Several candidates were identified and parsed into fragments to generate a library of fragments for each candidate. These candidate fragments were then evaluated using a pairwise comparison against segments of the polypeptide sequence of the protein of interest in order to identify suitable replacement fragments. Divergent protein sequences were then generated using these replacements. These constructs were validated by screening for enzymatic activity using B. subtilis and B. licheniformis as expression systems. FIG. 3 shows six pairwise alignments of a replacement fragment and the original corresponding segment of the protein of interest, selected from an exemplary divergent protein sequence which passed the validation screen. In brief, B. subtilis and B. licheniformis cells were engineered to express these constructs, and isolates were then evaluated using skim milk agar plates). In this case, each fragment was six amino acids in length. As shown by FIG. 3, the physiochemical properties of the six replacement fragments varied significantly with respect to the presence of hydrophobic or neutral polar amino acids, and with respect to total and net charge of the fragments. However, this divergent protein sequence was found to maintain enzymatic activity. This particular example illustrates the use of the present methods to design a single divergent protein sequence. However, it is understood that this general method, as well as the various other methods described herein, can be performed using any arbitrary protein (e.g., any enzyme) of interest. The methods and systems described herein thus represent a platform for the design and refinement of divergent protein sequences, without limitation to any particular organism or class of protein.

### EXAMPLES

### Example 1: Generation of a Divergent Amylase

A divergent amylase enzyme was generated using a method in accordance with the present disclosure. In this case, the PDB structure 4UZU was used as the protein of interest and evaluated as described by steps 102-110 of the method shown in FIG. 1. At step 112, 12 high-scoring template fragments were selected as replacement fragments based upon the mean Euclidean distance of atoms in the template fragment compared to atoms in the corresponding fragment being replaced. Each of these replacement fragments had an edit distance >3, which refers to the minimum number of operations required to transform the amino acid sequence of the original fragment into the amino sequence of the replacement fragment. The replacement fragments were used to generate divergent protein sequences, as described in step 114, and recombinant amylase proteins with these replacement fragments were constructed (using SEQ ID NO:1 as the baseline sequence) by site-directed mutagenesis. The resulting proteins were tested for expression in B. licheniformis. Activity was measured on a modified starch substrate (Megazyme Product code: S-RSTAR). TABLE 1 shows which blocks were found to be active for 8 tested substitutions. Positive clones were identified which expressed several divergent variants of the protease of interest, validating the methods described herein.

Results of Example 1.

| Enzyme | Starting | Original | Substituted | | |
|---|---|---|---|---|---|
| Class | Amino Acid | Amino Acids | Amino Acids | Score* | Active |
| Amylase | 83 | SAIAEL | RALAQL | 4.7 | Yes |
| Amylase | 125 | RETSGD | VMTNQE | 24.8 | Yes |
| Amylase | 163 | WDESRK | WDPSTE | 13.9 | Yes |
| Amylase | 294 | YASNSN | ADSSYL | 33.9 | Yes |
| Amylase | 411 | WTREGE | CQLFPG | 31.4 | Yes |
| Amylase | 79 | AQLKSA | SFGNDY | 6.9 | No |
| Amylase | 294 | YASNSN | RYNLAI | 41.9 | No |
| Amylase | 86 | AELHKQ | IRELAA | 6.1 | No |

*Score refers to the mean Euclidean distance between the two fragments after a rigid optimal alignment Table 1.

### Example 2: Generation of a Divergent Protease

A divergent protease enzyme was generated using a method in accordance with the present disclosure. In this case, the PDB structure 1ST3 was used as the protein of interest and analyzed in a similar manner to the protocol described above in Example 1. Site-directed mutagenesis was performed on SEQ ID. NO: 2 to generate recombinant variants for testing. Protease activity was determined using a modified casein substrate (Megazyme Product code: S-AZCAS). TABLE 2 shows which blocks were found to be active for 6 tested substitutions. Positive clones were identified which expressed several divergent variants of the protease of interest, validating the methods described herein.

Results of Example 2.

| Enzyme | Starting | Original | Substituted | | |
|---|---|---|---|---|---|
| Class | Amino Acid | Amino Acids | Amino Acids | Score* | Active |
| Protease | 13 | APAAHN | MLRILD | 7.1 | Yes |
| Protease | 42 | NIRGGA | KELGSE | 32.1 | Yes |
| Protease | 47 | ASFVPG | EIFDGL | 25.1 | Yes |
| Protease | 50 | VPGEPS | IVNDIR | 22 | Yes |
| Protease | 242 | NHLKNT | ATIAQL | 7.5 | Yes |
| Protease | 242 | NHLKNT | AIKTYL | 7.5 | No |

*Score refers to the mean Euclidean distance between the two fragments after a rigid optimal alignment Table 2.

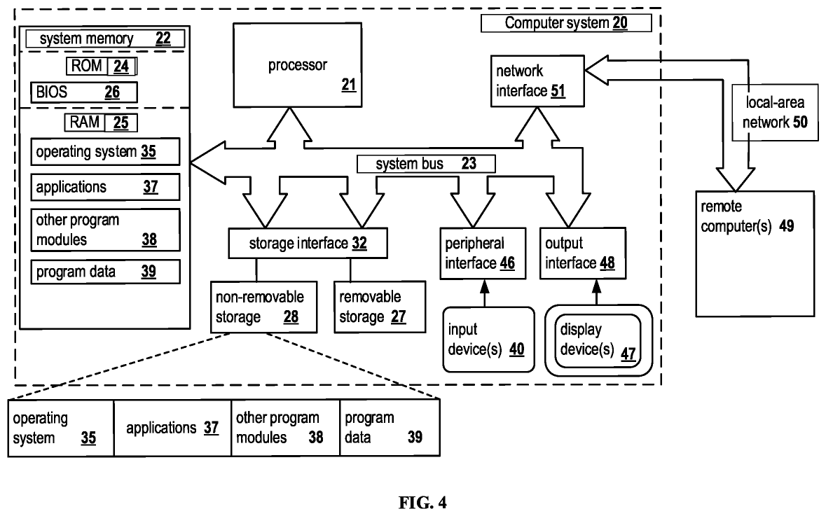

FIG. 4 is a block diagram illustrating a computer system 20 on which aspects of systems and methods for generating divergent protein sequences may be implemented in accordance with an exemplary aspect. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a graphics processing unit (GPU), a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™ InfiniBand™, Serial ATA, I2C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in FIGS. 1-2 may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made the specific goals will vary for different implementations. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

Sequence Listing

| SEQ ID NO. | SEQUENCE |
|---|---|
| 1 | ASTNGTMMQYFEWYLPNDGNHWTRLQNDAANLSSKGVTAVWIPPAYKGTSQNDVGYGA |
| | YDLYDLGEFNQKGTVRTKYGTKAQLKSAIAELHKQNIHVYGDVVMNHKGGADFTETVT |
| | AVEVDPNNRNRETSGDYQISAWTGFTFPGRGNTYSPFKWNWYHFDGTDWDESRKINRI |
| | YKFRGTGKAWDWEVSSENGNYDYLMYADIDYEHPDVINEMKKWGTWYAKELNLDGFRL |
| | DAVKHIKHDYLRDWVTHVRNQTGKELFTVAEYWQNDIGQLNNYLAKTNYNQSVFDAPL |
| | HYHFHYASNSNGNYDMRNILKGTVMESNPRLAVTLVENHDSQPGQSLESVVSTWFKPL |
| | AYAFILTRAEGYPSVFYGDYYGTKGNSNYEIPSLKTQLDPILTARKNYAYGLQRDYID |
| | HWDIIGWTREGEAARDKSGLATLITDGPGGYKWMEVGKQNAGEVWYDITGNRKETVTI |
| | NNDGWGQFYVNGGSVSIYVQK |
| 2 | AEEAKEKYLIGFNEQEAVSEFVEQVEANDEVAILSEEEEVEIELLHEFETIPVLSVEL |
| | SPEDVDALELDPAISYIEEDAEVTTMAQSVPWGISRVQAPAAHNRGLTGSGVKVAVLD |
| | TGISTHPDLNIRGGASFVPGEPSTQDGNGHGTHVAGTIAALNNSIGVLGVAPSAELYA |
| | VKVLGADGEGAISSIAQGLEWAGNNGMHVANLSLGSPSPSATLEQAVNSATSRGVLVV |
| | AASGNSGASSISYPARYANAMAVGATDQNNNRASFSQYGAGLDIVAPGVNVQSTYPGS |
| | TYASLNGTSMATPHVAGAAALVKQKNPSWSNVQIRNHLKNTATSLGSTNLYGSGLVNA |
| | EAATR |

1. A computer-implemented method for generating divergent protein sequences, comprising:
a) receiving structural data for a protein of interest;
b) generating a first library of fragments using the structural data, wherein the first library of fragments comprises fragments of the protein of interest;
c) selecting one or more template proteins;
d) generating a second library of fragments, wherein the second library of fragments comprises fragments of each of the one or more template proteins;
e) comparing at least one fragment in the first library of fragments against one or more fragments in the second library of fragments;
f) selecting a replacement fragment for at least one of the fragments in the first library of fragments, based on the comparison; and
g) generating a divergent protein sequence, wherein the divergent protein sequence comprises at least one replacement fragment.

2. The method of claim 1, wherein the structural data comprises a three-dimensional structure of the protein of interest.

3. The method of claim 1, wherein the structural data comprises a protein data bank (PDB) file containing coordinates representing a three-dimensional structure of the protein of interest.

4. The method of claim 3, wherein the first library of fragments is generated by parsing the structural data into a series of segments and extracting coordinates for each segment from the structural data.

5. The method of claim 4, wherein the segments comprise at least, at most, or exactly 2, 3, 4, 5, 6, 7, 8, 9, 10, 11, 12, 13, 14, 15, 16, 17, 18, 19, or 20 amino acids in length.

6. The method of claim 1, wherein the first library of fragments comprises fragments of a uniform length.

7. The method of claim 1, wherein the first library of fragments comprises fragments of at least, at most, or exactly 2, 3, 4, 5, 6, 7, 8, 9, 10, 11, 12, 13, 14, 15, 16, 17, 18, 19, or 20 amino acids in length.

8. The method of claim 1, wherein the first library of fragments comprises fragments of at least, at most, or exactly 6 or 8 amino acids in length.

9. The method of claim 1, wherein the first library of fragments comprises coordinates representing a three-dimensional structure for each of a plurality of fragments of the protein of interest.

10. The method of claim 1, wherein the one or more template proteins comprise proteins for which a crystal structure is available.

11. The method of claim 1, wherein the one or more template proteins are selected based upon one or more parameters, comprising:
a) a sequence identity threshold parameter;
b) an enzyme classification parameter;
c) the presence of one or more protein domains; and/or
d) a superimposition parameter reflecting a degree of local or global fit when a 3D structure of the template protein, or a portion thereof, is superimposed on a 3D structure of the protein of interest, or a portion thereof.

12. The method of claim 1, wherein the one or more template proteins are selected based upon a maximum sequence identity threshold, wherein the maximum sequence identity comprise at most 10, 20, 30, 40 50, 60, 70, 80, or 90% full length sequence identity compared to the protein of interest.

13. The method of claim 1, wherein structural data is provided for each of the template proteins and the second library of fragments is generated by parsing the structural data for each of the template proteins into a series of segments and extracting coordinates for each segment from the structural data.

14. The method of claim 13, wherein the segments comprise at least, at most, or exactly 2, 3, 4, 5, 6, 7, 8, 9, 10, 11, 12, 13, 14, 15, 16, 17, 18, 19, or 20 amino acids in length.

15. The method of claim 1, wherein the second library of fragments comprises fragments of a uniform length.

16. The method of claim 1, wherein the second library of fragments comprises fragments of at least, at most, or exactly 2, 3, 4, 5, 6, 7, 8, 9, 10, 11, 12, 13, 14, 15, 16, 17, 18, 19, or 20 amino acids in length.

17. The method of claim 1, wherein the comparing step comprises generating a pairwise alignment score for at least one fragment in the first library of fragments against one or more of the fragments in the second library of fragments.

18. The method of claim 17, wherein the comparing step comprises generating a pairwise alignment score for each fragment in the first library of fragments against each fragment in the second library.

19. The method of claim 17, wherein the pairwise alignment score is based on a three-dimensional alignment of the fragment in the first library of fragments against the respective fragment in the second library of fragments.

20. The method of claim 19, wherein pairwise alignment score is based on a three-dimensional alignment of the backbone atoms of the aligned fragments.

21. The method of claim 19, wherein pairwise alignment score is based on a three-dimensional alignment of the backbone and side chain atoms of the aligned fragments.

22. The method of claim 1, wherein replacement fragments are selected for fragments in the first library of fragments based upon pairwise alignment scores, wherein each pairwise alignment score compares the three-dimensional alignment of the fragment in the first library against a fragment in the second library of fragments.

23. The method of claim 1, wherein the replacement fragment is a fragment selected from the second library of fragments which displays the highest pairwise alignment score compared against the respective fragment in the first library of fragments.

24. The method of claim 1, further comprising generating a predicted protein structure for the divergent protein sequence.

25. The method of claim 24, further comprising generating a model quality score for the predicted protein structure.

26. A system for generating divergent protein sequences, comprising a processor configured to:
a) receive structural data for a protein of interest;
b) generate a first library of fragments using the structural data, wherein the first library of fragments comprises fragments of the protein of interest;
c) select one or more template proteins;
d) generate a second library of fragments, wherein the second library of fragments comprises fragments of each of the one or more template proteins;
e) compare at least one fragment in the first library of fragments against one or more fragments in the second library of fragments;
f) select a replacement fragment for at least one of the fragments in the first library of fragments, based on the comparison; and
g) generate a divergent protein sequence, wherein the divergent protein sequence comprises at least one replacement fragment.

27. The system of claim 26, wherein the processor is further configured to perform the method of claim 1.

28. A non-transitory computer-readable medium storing thereon computer-executable instructions for generating divergent protein sequences, comprising instructions for:
a) receiving structural data for a protein of interest;
b) generating a first library of fragments using the structural data, wherein the first library of fragments comprises fragments of the protein of interest;
c) selecting one or more template proteins;
d) generating a second library of fragments, wherein the second library of fragments comprises fragments of each of the one or more template proteins;
e) comparing at least one fragment in the first library of fragments against one or more fragments in the second library of fragments;
f) selecting a replacement fragment for at least one of the fragments in the first library of fragments, based on the comparison; and
g) generating a divergent protein sequence, wherein the divergent protein sequence comprises at least one replacement fragment.

29. The non-transitory computer-readable medium of claim 28, further comprising instructions for performing the method of claim 1.

30. A divergent protein sequence produced by a computer, comprising a processor configured to:
a) receive structural data for a protein of interest;
b) generate a first library of fragments using the structural data, wherein the first library of fragments comprises fragments of the protein of interest;
c) select one or more template proteins;
d) generate a second library of fragments, wherein the second library of fragments comprises fragments of each of the one or more template proteins;
e) compare at least one fragment in the first library of fragments against one or more fragments in the second library of fragments;
f) select a replacement fragment for at least one of the fragments in the first library of fragments, based on the comparison; and
g) generate the divergent protein sequence, wherein the divergent protein sequence comprises at least one replacement fragment;
wherein the divergent protein sequence shares at most 10% full-length sequence identity with the sequence of the protein of interest.

31. The divergent protein sequence of claim 30, wherein the divergent protein sequence shares at most 20, 30, 40, 50, 60, 70, 80, or 90% full-length sequence identity with the sequence of the protein of interest.

32. The divergent protein sequence of claim 30, wherein the divergent protein sequence shares at most 10, 20, 30, 40, 50, 60, 70, 80, or 90% full-length sequence identity with the sequence of the protein of interest and the protein of interest comprises an enzyme; and
wherein the divergent protein sequence encodes a protein that maintains at least substantially equivalent enzymatic activity compared to the protein of interest.
